(19) United States
(12) Patent Application Publication
(10) Pub. No.: US 2017/0035287 A1
(43) Pub. Date: Feb. 9, 2017

(54) DYNAMIC SURGICAL DATA OVERLAY

(71) Applicant: Novartis AG, Basel (CH)

(72) Inventors: Hugang Ren, Irvine, CA (US); Lingfeng Yu, Rancho Santa Margarita, CA (US)

(21) Appl. No.: 14/817,348

(22) Filed: Aug. 4, 2015

(51) Int. Cl. A61B 3/00; G09G 5/38; G06T 7/00; G09G 5/377; A61B 3/10; G06K 9/32

(52) U.S. Cl. CPC A61B 3/0058; A61B 3/102; G06K 9/3233; G06T 7/0012; G09G 5/377; G09G 5/38; G06T 2207/10101; G06T 2207/20212; G06T 2207/30041; G09G 2380/08; G09G 2340/12

(57) ABSTRACT

A method for display optimization includes receiving an image of a surgical site from an imaging system. The method further includes determining a region of interest at a first location within the image. The method further includes generating a surgical data overlay at a first position, the first position associated with the first location of the region of interest. The method further includes detecting that the region of interest has moved to a second location within the image. The method further includes, in response to detecting that the region of interest has moved to the second location, moving the surgical data overlay to a second position, the second position associated with the second location. The method further includes displaying the image and surgical data overlay to a user.

### TECHNICAL FIELD

The present disclosure is directed to methods and systems for ophthalmic medical procedures, and more particularly, to methods and systems involving imaging for such procedures.

### BACKGROUND

Many microsurgical procedures require precision cutting and/or removal of various body tissues. For example, Internal Limiting Membrane (ILM) removal and epi-retinal membrane (ERM) removal are useful surgical treatments of different macular surface diseases. However, the surgical techniques for ILM and ERM peeling require skill and patience. Precise and carefully constructed surgical instruments are used for each segment of the surgical technique.

ILM and ERM procedures use a two-step technique. The first step includes gaining an edge of the membrane and the second step includes grasping and peeling the membrane. Some operators use a scraper to gain the edge of the membrane. The operator gently scrapes the membrane to separate membrane edges so that an edge is ready to be grasped. Next, the operator introduces a special forceps to grasp and peel the membrane. However, because each step requires patience and precision, an operator may sometimes scrape and then attempt to grasp the tissue multiple times during a single surgical procedure.

To aid the operator with these types and other types of surgical procedures, operators may use an imaging system that presents a microscope view of the tissue to be treated, such as tissue of the patient's eye. Accordingly, the user of such an imaging system may be provided with a close-up view of the surgical instruments, such as forceps or other tools, as well as the region of the eye that is of interest. In some cases, the operator may also be provided with additional information that may be useful to the operator. For example, the operator may be provided with an Optical Coherence Tomography (OCT) image of the region of the eye that is of interest. OCT imaging generally utilizes near-infrared light and is able to obtain or generate images of tissue beneath the surface. There is a need for continued improvement in the use and operability of surgical systems and tools for various ophthalmic procedures.

### SUMMARY

A method for display optimization includes receiving an image of a surgical site from an imaging system. The method further includes determining a region of interest at a first location within the image. The method further includes generating a surgical data overlay at a first position, the first position associated with the first location of the region of interest. The method further includes detecting that the region of interest has moved to a second location within the image. The method further includes, in response to detecting that the region of interest has moved to the second location, moving the surgical data overlay to a second position, the second position associated with the second location. The method further includes displaying the image and surgical data overlay to a user.

A system includes an imaging module to obtain an image of a surgical site. The system further includes a display module to display the image of the surgical site to a user and display surgical data overlaying the image of the surgical site. The system further includes a tracking module to determine a region of interest of the surgical site at a first location. The system further includes a control module to detect that the region of interest has moved to a second location based on data from the tracking module and instruct the display module to move the surgical data to a new position over the image based on the new region of interest.

A method for display optimization includes receiving an image of a surgical site from an imaging system. The method further includes determining a region of interest within the image. The method further includes generating a surgical data overlay at a first position in the image, the first position associated with a first location of the region of interest. The surgical data overlay includes an Optical Coherence Tomography (OCT) image of the region of interest. The method further includes detecting that the region of interest has moved to a second location. The method further includes, in response to detecting that the region of interest has moved to the second location, determining a second position for the surgical data overlay in the image based on both user preferences and the second location of the region of interest. The method further includes displaying the image and surgical data overlay at the second position to a user.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a diagram showing an illustrative ophthalmic surgical system.

FIG. 2 is a diagram showing an illustrative image of a patient's eye as may be seen through an imaging system during a surgical procedure.

FIG. 3 is a flowchart showing an illustrative method for providing a dynamic surgical data overlay.

FIGS. 4A, 4B, and 4C are diagrams showing illustrative surgical data overlays that are dynamically placed based on a region of interest.

FIGS. 5A, 5B, 5C, and 5D are diagrams showing illustrative surgical data overlays that are dynamically placed based on a region of interest and user preferences.

FIG. 6 is a diagram showing an image system that uses tool tracking to determine a current region of interest.

FIG. 7 is a diagram showing an image system that uses eye tracking to determine a current region of interest.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

The present disclosure is directed to methods and systems for displaying surgical data along with a standard image of a surgical site. In various procedures, a user may observe a region of interest, such as a particular tissue region at a surgical site, using an imaging system. The imaging system may also display additional surgical data to the user aside from the image of the region of interest. In one example, the additional surgical data includes an OCT image. For example, some imaging systems include a microscope imaging system and an OCT imaging system. The OCT imaging system obtains an OCT image that includes a cross-sectional view of the region of interest. Thus, the OCT image may be used to visualize tissue below the outer surface tissue. In some cases, the OCT image is provided as a surgical data overlay within the microscope image.

Such an imaging system permits a user to observe both a conventional microscope image and an OCT image while using a surgical instrument to perform an ophthalmic surgical procedure such as an ILM removal. The conventional microscope image is observed using light that is within the visible spectrum having a wavelength ranging between about 400 nanometers and 700 nanometers. The OCT image is usually generated using light in the near infrared range having a wavelength within a range of about 700 nanometers to 2600 nanometers. It is, however, also possible to obtain OCT images using light in the visible spectrum range. Thus, an OCT image may be obtained using light within any practicable wavelength range.

Generally, such surgical data overlays remain fixed in a viewable location with respect to the image with which they are included. Thus, as the user directs his or her attention to different regions of interest of the surgical site within the image, the user has to direct his or her vision away from those regions of interest to view the surgical data overlay. This can be risky if the user is in the middle of a delicate procedure. The user may have to hold the tools steady while redirecting attention to the surgical data overlay.

According to principles described herein, the present disclosure is directed to dynamically modifying the position of the surgical data overlay in real time. Through various mechanisms, the current region of interest is determined. The region of interest refers to the general area at which a user is generally directing his or her attention. One example of a mechanism that can be used to determine the region of interest is an eye tracking mechanism that tracks where within the image the user's eyes are directed. Other examples, which will be discussed in further detail below, include tool tracking and OCT beam detection. After the current region of interest has been determined, the position of the surgical data overlay can be changed accordingly. Specifically, if the region of interest moves to a new location, the surgical data overlay can be repositioned to be near that new location.

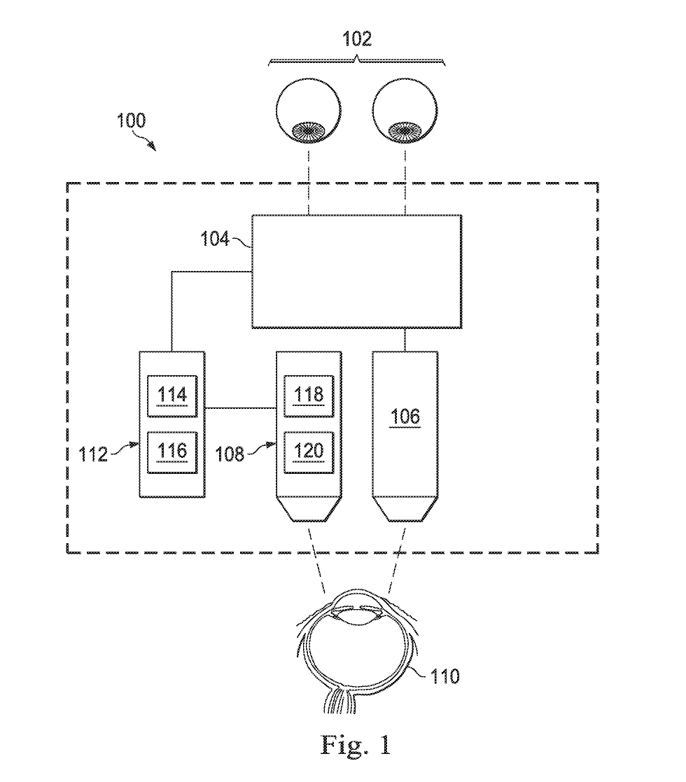

FIG. 1 is a diagram showing an illustrative ophthalmic imaging system 100. According to the present example, the ophthalmic imaging system 100 includes an image viewer 104, a microscope imaging system 106, an OCT imaging system 108, and a control system 112. The ophthalmic imaging system 100 provides a user 102 with a microscope view and an OCT image of the region of interest within a target region of the patient's body. In this example, the target region is an eye 110 of the patient.

The microscope imaging system 106 obtains images of the patient's eye 110 using light within the visible spectrum. The visible spectrum defines the wavelength range of light that is visible to the human eye. The visible spectrum includes electromagnetic radiation having a wavelength that is, as indicated above, generally within a range of about 400 nanometers to 700 nanometers, though this wavelength range may vary slightly for different individuals. The microscope imaging system 106 may use a system of lenses to provide a close-up view of the patient's eye 110 or even a specific region of interest within the patient's eye 110. Such an image may then be provided to the image viewer 104.

The OCT imaging system 108 obtains OCT images of the patient's eye 110. It uses various techniques to obtain depth resolved images of the patient's tissue beneath the surface of the tissue that are not able to be obtained from the use of a standard microscope. This is done using coherence gating based on light that is within the OCT spectrum. As indicated above, this range includes electromagnetic radiation having a wavelength between about 700 nanometers and 2600 nanometers, and in some cases can be extended to the visible light range of about 400 nanometers to 700 nanometers. By using coherence gating, the OCT imaging system 108 can display an image of tissue below the surface tissue and generate a cross-sectional view of such tissue. As such, the OCT imaging system 108 may be used to obtain a cross-sectional view of the region of interest at which the user 102 is operating. A benefit of this is that the user 102 is able to see how interactions between the surgical instrument and the surface of an ILM affect the tissue below the surface of the ILM. Specifically, the user 102 can use the cross-sectional image to help avoid accidental damage to the underlying retina. In some examples, the OCT imaging system 108 is integrated with the conventional microscope imaging system 106. In some examples, however, the OCT imaging system 108 may be a separate apparatus that provides the OCT images to the image viewer 104.

The OCT imaging system 108 includes various components that are used to perform the OCT imaging function. For example, the OCT imaging system 108 may include an OCT light source 118 to project an OCT beam at a region of interest. The OCT imaging system 108 may also include an OCT capture device 120 that detects OCT light reflected from the region of interest. The OCT imaging system 108 then uses the information obtained by the OCT capture device 120 to construct an image of the region of interest. In some examples, the image may be a two-dimensional cross-section of the region of interest that provides a view beneath the surface of tissue within the region of interest. In some examples, the image may be a three-dimensional image that also provides a three-dimensional view beneath the surface.

The image viewer 104 displays to a user 102 or other operator, the images obtained by both the microscope imaging system 106 and the OCT imaging system 108. The image viewer 104 may display the images in a variety of ways, such as on a monitor, display screen, on the microscope eyepiece, or in other ways. In some examples, the microscope imaging system 106 may provide stereoscopic images formed of at least two images. The image viewer 104 may display the at least two images to different eyes of the user 102, thus creating a three dimensional effect.

The control system 112 is a computing system that may process images obtained from the OCT imaging system 108. The control system 112 may track the user's region of interest to determine the optimal position of a surgical data overlay such as an OCT image. In some examples, the control system 112 may be integrated with the image viewer 104. In some examples, the control system 112 is a discrete component that is separate from, and in communication with, the image viewer 104 and the OCT imaging system 108.

The control system 112 also includes a processor 114 and a memory 116. The memory 116 may include various types of memory including volatile memory (such as Random Access Memory (RAM)) and non-volatile memory (such as solid state storage). The memory 116 may store computer readable instructions, that when executed by the processor 114, cause the control system 112 to perform various functions, including the repositioning of the surgical data overlay as described herein. The memory 116 may also store data representing images captured by the imaging systems 106, 108 as well as modified versions of those images.

In some examples, the OCT imaging system 108 may be an endoprobe. An endoprobe is a device that is designed to be inserted into an orifice of a patient and is used to view patient tissue. It may be used to diagnose various diseases or conditions. Once an OCT image is acquired from such an endoprobe, a surgical data overlay may be provided and positioned such that it follows the user's region of interest.

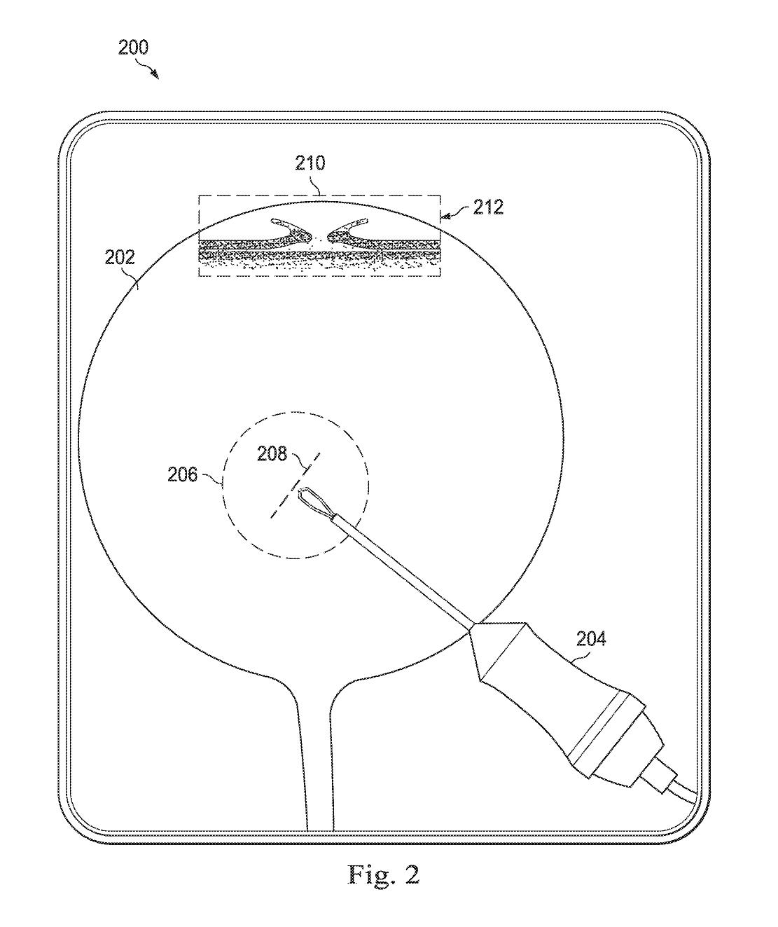

FIG. 2 is a diagram showing an illustrative combined microscope image and surgical data overlay view 200 of a patient's eye as presented or displayed by the image viewer 104. According to the present example, the image viewer 104 (e.g., 104, FIG. 1) overlays a surgical data overlay 210, such as an OCT image 212, on a microscope image 202. Thus, the user can view a potential region of interest 206 along with the surgical instrument 204 being used to operate within the region of interest 206. The dotted line 208 in FIG. 2 represents the cross-sectional line at which the cross-sectional OCT image 212 in the surgical data overlay 210 is taken. Thus, as may be seen, the image viewer 104 projects the OCT image 212 onto the microscope image 202 in a manner permitting the user to visually observe both images 202, 212 at once in real time.

While the example illustrated in FIG. 2 and other examples described herein relate to a surgical data overlay 210 that presents an OCT image 212, other types of information may be provided within the surgical data overlay 210. For example, instead of a real-time OCT image view, the surgical data overlay 210 may provide a still OCT image of the patient's eye. In some cases, such an OCT image may be enhanced to more clearly show certain features. For example, an enhanced image may indicate the thickness of an ERM and where the ERM is attached. An enhanced image may emphasize the internal limiting membrane (ILM). An enhanced image may emphasize sub-retinal fluid (SRF), the thickness of the SRF and/or the volume of the SRF. The surgical data overlay 210 may also display various pathological data. In some examples, the surgical data overlay may include images of hand-drawn graphs. Other types of information that may be useful to a user of the imaging system are contemplated as well. For example, the other types of information may include surgical parameters, a thickness of one or more retinal layers, flow velocity of one or more retinal vessels, retinal angiographic information, and characteristic information of one or more retinal layers.

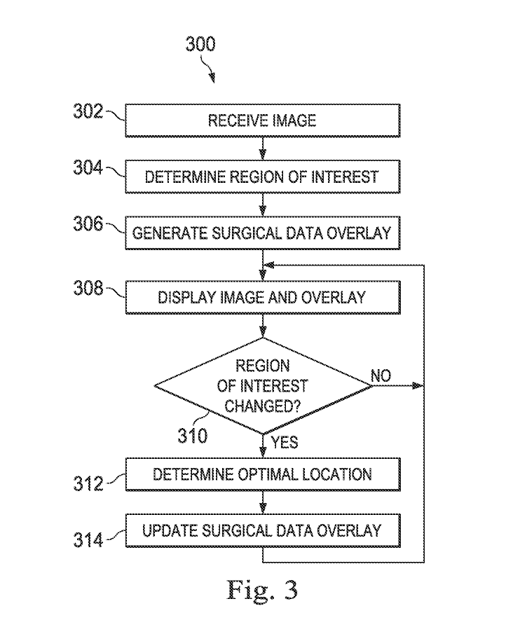

FIG. 3 is a flowchart showing an illustrative method 300 for providing a dynamic surgical data overlay. In some examples, the method 300 is performed by a control system (e.g., 112, FIG. 1). According to the present example, the method 300 includes a step 302 for receiving an image from an imaging system (e.g., 106, FIG. 1). The image may be of a surgical site such as a retina. The surgical site within the image may have several locations at which a user of the imaging system may perform surgical activities for treatment.

The method 300 further includes a step 304 for determining a region of interest within the image. The region of interest indicates a specific region within the image at which the user's attention is currently directed. For example, if a user is performing a particular treatment activity at a particular location within the image, then that location may be designated as the current region of interest.

The region of interest within the image may be determined through a variety of mechanisms. In one example, the region of interest is determined by determining where a surgical tool within the image is located. In one example, the region of interest is determined based on the location at which the user's eyes are currently directed. In one example, the region of interest is determined by detecting where within the image an OCT beam is being directed.

The location and/or orientation of a tool, such as a forceps, can be used to determine the user's region of interest. In one example, the location of a specific portion of a tool within the image can be used to identify the current region of interest. In the case of a forceps, the specific portion of the tool may be the tip of the forceps. Other regions of the tool might also be used. Thus, the region of interest can be determined based on the location of the tip of the forceps.

Various mechanisms can be used to determine the location and/or orientation of the tool with respect to the image. In some examples, the tool may have location or orientation sensing devices attached thereto or embedded within that can detect such information. For example, the tool may have a gyroscope, accelerometer, or other type of sensor associated therewith to determine the current orientation. In some other examples, however, the location and/or orientation may be determined by analysis of the image itself Specifically, the control system may apply a function that detects the boundaries of the tool within the image. The control system may also apply a function to detect locations within the surgical site. Thus, the location of the tool with respect to the surgical site can be determined. Other arrangements may use a combination of detector inputs and analysis for detection. Still other arrangements and systems are also contemplated.

In some examples, a tool may include markers, engravings, or other indicators that help identify the location of the tool with respect to the surgical site. In some implementations, the function used to analyze the image is configured to detect such markers or engravings. In one example, the marker may be a colored portion of the tool. The color or nature of the marker may be such that the portion is easily recognizable by the function that analyzes the image. Other examples may employ surface structures, designs, color contrasts, or other markers that are recognizable by the function.

In some examples, a tool tracking system can determine the general location in which a tool is operating over a set period of time. Presumably, during a surgical operation, the tool will be moving as the operator of the tool performs the associated surgical operations within that tool. The tool tracking system can then determine a region of interest that encompasses the general area in which the tool has been moving during the past set period of time. The period of time can be selected so as to obtain enough tracking data to determine an acceptable region of interest but not so long that there is an undesired delay when the user moves the tool to a new location and thus moves the region of interest to a different location within the image. In some examples, the period of time may be manually set by the user. It may be, for example, one second, five seconds, or within a range of 0-20 seconds. Larger and smaller times are also contemplated.

In some examples, eye tracking may be used to determine the current region of interest. For example, an eye tracking system may scan the user's eyes to determine the location within the image at which the user's eyes are directed. Presumably, such a location corresponds to the area of the image at which the user is most interested in seeing, and may include the area at which the user is currently performing surgical operations.

In some examples, an eye tracking module, as will be described in further detail below, can determine the general location in the image at which a user's eyes are directed over a set period of time. Presumably, during a surgical operation, the user will be viewing various locations near the region at which he or she is operating. The control system can then determine a region of interest that encompasses the general area at which the user's eyes have been directed over the past set period of time. The period of time can be selected so as to obtain enough tracking data to determine an acceptable region of interest but not so long that there is an undesired delay when the user moves his or her eyes to a new location and thus moves the region of interest to a different location within the image. In some examples, the period of time may be manually set by the user. In some examples, the control system may filter out tracking data that corresponds to the user viewing the surgical data overlay. If the user looks away from the region of interest to view the nearby surgical data overlay, it may be desirable not to include such tracking data in order to avoid biasing the region of interest towards the surgical data overlay.

In some examples, the location of the surgical site at which an OCT beam is directed can be used to identify the current region of interest. As described above, the surgical data overlay may include an OCT image. Such an image is obtained by directing an OCT beam at the surgical site. Then, the OCT image capture device (e.g. 120, FIG. 1) detects OCT light reflected from beneath the surface of the surgical site. Generally, the region of interest at which the OCT beam is directed will correspond to where the user is performing a surgical operation and is thus a region of interest.

Various mechanisms may be used to determine where the OCT beam is being directed. In one example, a tracking system associated with OCT image device may be used to determine where within the image the OCT beam is being directed. In some examples, an analysis of the image obtained by the microscope imaging system may be performed to determine where the OCT beam is being directed. While OCT light may not be readily identifiable to the human eye, an analysis of the image may be able to detect the location within the image at which OCT light is being directed.

In some cases, the control system may be configured to take into account data from multiple sources to determine the region of interest. For example, the control system may receive tool tracking data, eye tracking data, OCT beam position data, and/or other data. All such forms of data can be used to determine the region of interest.

The method 300 further includes a step 306 for generating the surgical data overlay. As described above, the surgical data overlay may include various types of information including, for example, a real time OCT image of the surgical site, surgical or instrument data, patient data, sensed data relating to the patient's physiological condition, or other information. In some examples, the surgical data overlay may include a still OCT image of the surgical site. In the case where the surgical site is an eye of the patient, the still OCT image may be enhanced to emphasize, through highlighting, increased image intensity, or other techniques, various features such an epi-retinal membrane (ERM), a thickness of the ERM, and a contour of the ERM.

The position of the surgical data overlay is determined based on the location of the region of interest. Specifically, the position of the surgical data overlay is set so with respect to the region of interest. For example, the surgical data overlay may be positioned so it is directly adjacent to, such as above the region of interest.

At step 308, the control system displays the image and the surgical data overlay together. In one example, the control system provides the image to the image viewer for viewing by the user. Because the surgical data overlay is positioned so that it is near the current region of interest, the user does not have to look too far away from the region of interest to view the information contained within the surgical data overlay.

At step 310, the control system determines whether the region of interest has changed. Specifically, the control system determines whether the region of interest has moved to another location within the image. Such information may be based on tracking data obtained from a tool tracking system, an eye tracking system, or some other mechanism used to determine the current region of interest.

In some embodiments, the control system is configured to determine whether the region of interest has substantially changed location. While the region of interest may move slightly from its current position based on small movements in the tool or small movements in the user's eyes, such small movements may not merit a change in the position of the accompanying surgical data overlay. For example, if the region of interest moves less than a certain amount, such as 1 millimeter, then the surgical data overlay remains unchanged. Larger or smaller distances are contemplated.

If there has been no substantial change in the location of the region of interest, then the method returns to step 308, at which the control system causes the image view to display the image along with the surgical data overlay. But, if there has been a substantial change in the region of interest, then the method proceeds to step 312.

At step 312, the method includes determining an optimal location of the surgical data overlay. Such a determination is based on the new location of the region of interest. As will be described in further detail below, the optimal location may also take into account various user preferences.

The method 300 further includes a step 314 for updating the position of the surgical data overlay based on the determined optimal position. The method then returns to step 308 at which the control system displays the image and the surgical data overlay at its new position. The control system continues to cause the surgical data overlay to be displayed at that new position until the region of interest again changes. At such a time, the location of the surgical data will be updated again accordingly.

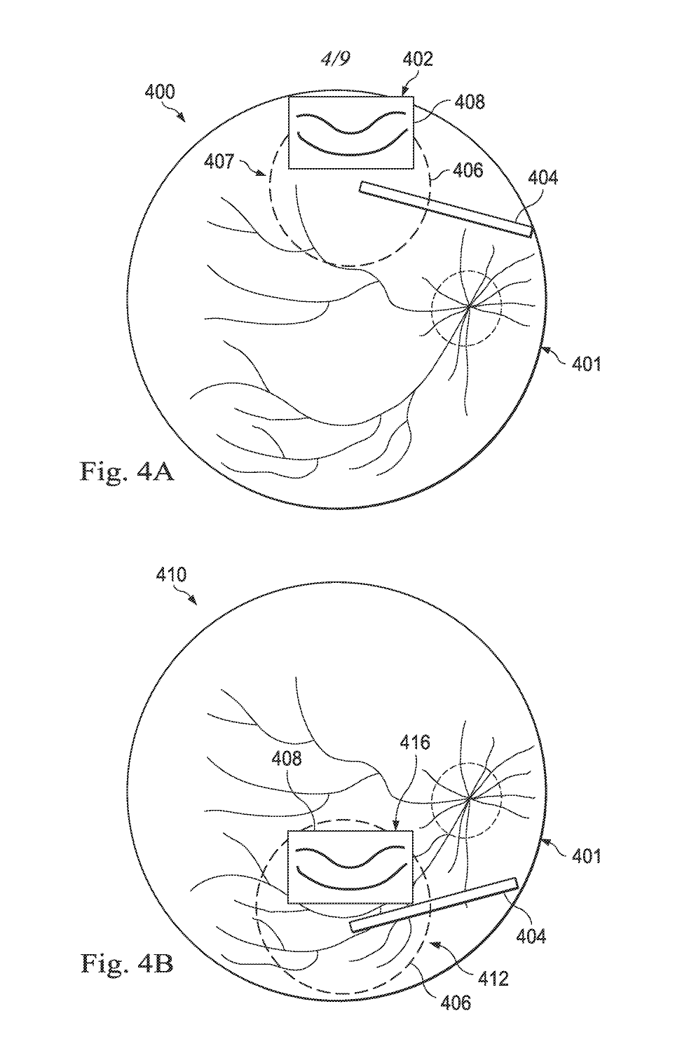

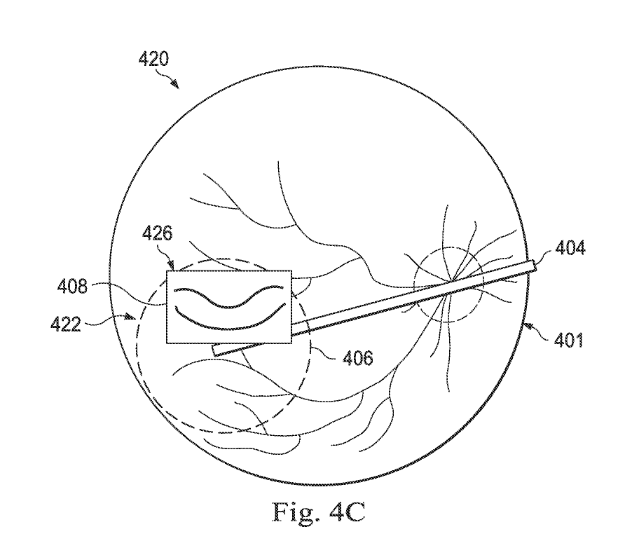

FIGS. 4A, 4B, and 4C are diagrams showing illustrative surgical data overlays 408 that are dynamically placed based on a current region of interest 406. FIG. 4A illustrates an image 400 of a surgical site 401. A surgical tool 404 is visible within the image 400. The image 400 includes a surgical data overlay 408 at a first position 402. The first position 402 is based on the current region of interest 406 within the surgical site 401. The location 407 of the region of interest 406 may have been determined based on the location of a tool 404 that is visible within the image 400, based on the region at which the user's eyes are directed as described above, or based on other information indicative of the user's area of focus.

FIG. 4B illustrates an image 410 of the surgical site 401 after the region of interest 406 has been moved to a different location 412. In one example, the region of interest 406 may have moved to the new location 412 in response to detecting that the user's eyes are directed at the new location 412. In one example, the region of interest 406 may have moved to the new location 412 in response to detecting that the tool 404 has moved to the new location 412. Because the region of interest 406 has moved to a new location 412, the surgical data overlay 408 has also moved to a new position 416. The new position 416 is based on the location 412 of the region of interest 406. Specifically, the new position 416 is near the top of the region of interest 406 at the new location 412.

FIG. 4C illustrates an image 420 of the surgical site 401 after the region of interest 406 has been moved to another different location 422 within the image. In one example, the region of interest 406 may have moved to the new location 422 in response to detecting that an OCT beam is now directed at the new location 422. Because the region of interest 406 has moved to a new location 422, the surgical data overlay 408 has also moved to a new position 426. The new position 426 is based on the location 422 of the region of interest 406. Specifically, the new position 426 is near the top of the region of interest 426 at the new location 422.

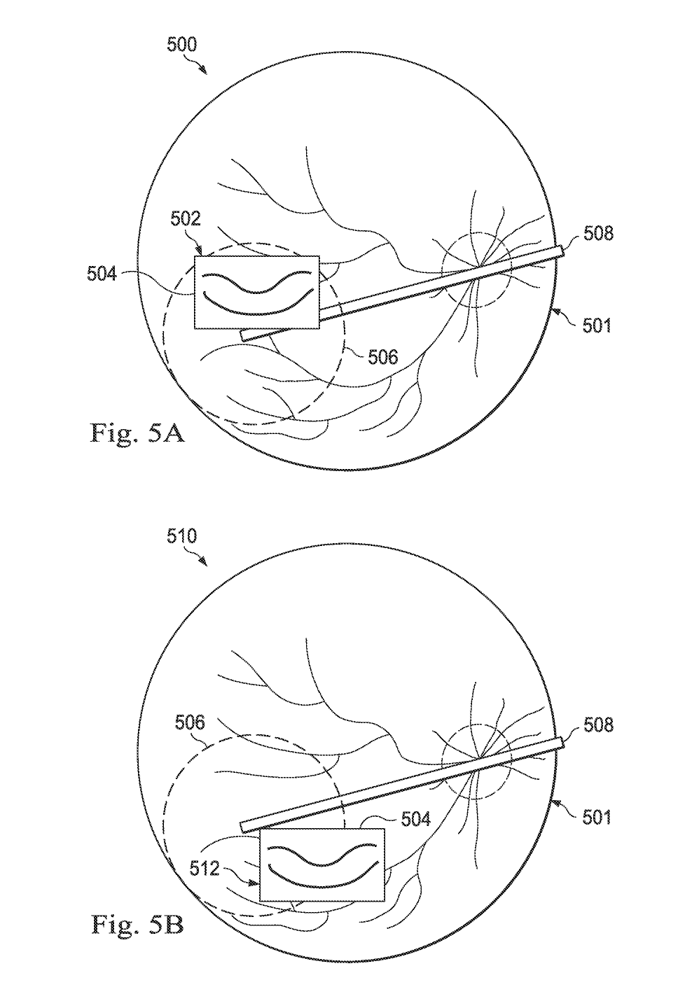

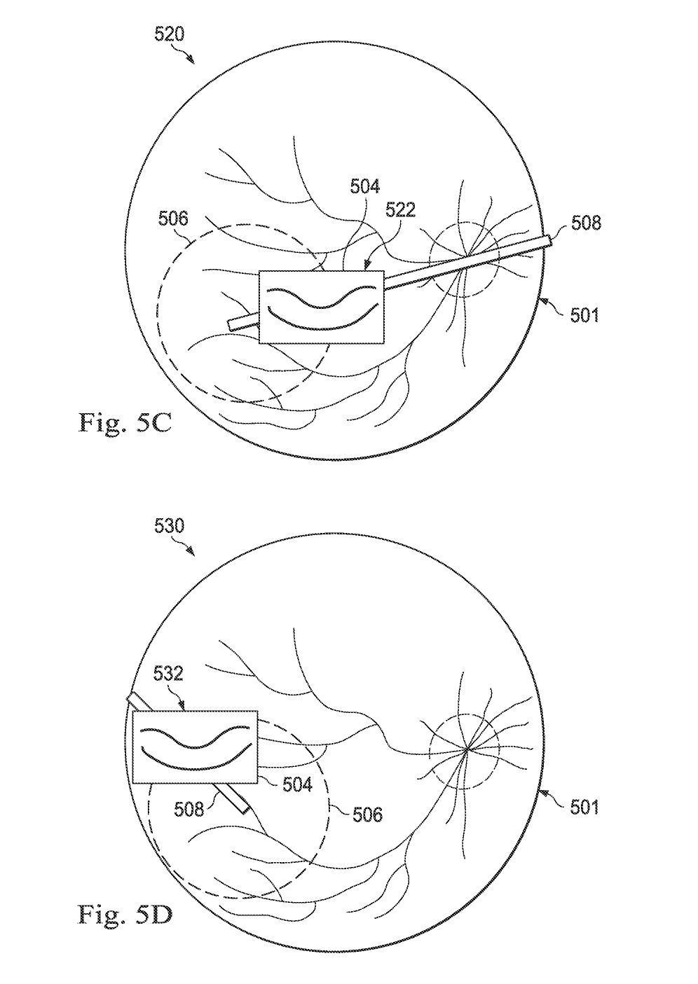

FIGS. 5A, 5B, 5C, and 5D are diagrams showing images with illustrative surgical data overlays that are dynamically placed based on a region of interest 506 and user preferences. In some examples, the control system may have a default setting for placement of the surgical data overlay 504 with respect to the region of interest 506. For example, the default setting may be to have the surgical data overlay 504 positioned near the top of the region of interest 506. But, some users may prefer other positions of the surgical data overlay 504 with respect to the region of interest 506. Thus, a user may have the ability to change the settings of the imaging system to display the surgical data overlay 504 at the desired position with respect to the region of interest 506.

FIG. 5A illustrates an image 500 of a surgical site 501 in which the surgical data overlay 504 is at a position 502 that is near the top of the region of interest 506. In this example, such a position 502 partially obstructs the tool 508. If the user knows that he or she typically operates the tool from a specific position, then the user may set the preferences through the control system so that the surgical data overlay is always at a specific position with respect to the region of interest 506. FIG. 5B illustrates an image 510 of the surgical site 501 in which the surgical data overlay 504 is at a position 512 at the bottom right side of the region of interest 506. FIG. 5C illustrates an image 520 of the surgical site 501 in which the surgical data overlay 504 is at a position 522 that is at the right side of the region of interest 506. FIG. 5D illustrates an image 530 of the surgical site 501 in which the surgical data overlay 504 is at a position 532 that is at the left side of the region of interest 506. Some user preferences that may be set or selected include, for example, whether to have the surgical data overlay to the top, bottom, left, or right of the center of the region of interest.

In some examples, the position of the surgical data overlay 504 with respect to the region of interest 506 may be determined dynamically based on a variety of factors. For example, if the user prefers that the surgical data overlay 504 not obstruct any portion of the tool 508, then the user may set the preferences such that the surgical data overlay 504 will be positioned such that it does not obstruct the tool 508 as shown in FIG. 5B. In some examples, however, a user may wish that the surgical data overlay 504 be positioned over a portion of the tool 508 while leaving the tip of the tool 508 unobstructed as shown in FIGS. 5C and 5D. Thus, the user can change the settings accordingly to provide such functionality. Thus, as the user moves the tool 508 to various positions within the region of interest, the surgical data overlay 504 may follow the tool in a manner that still leaves the tip of the tool 508 exposed.

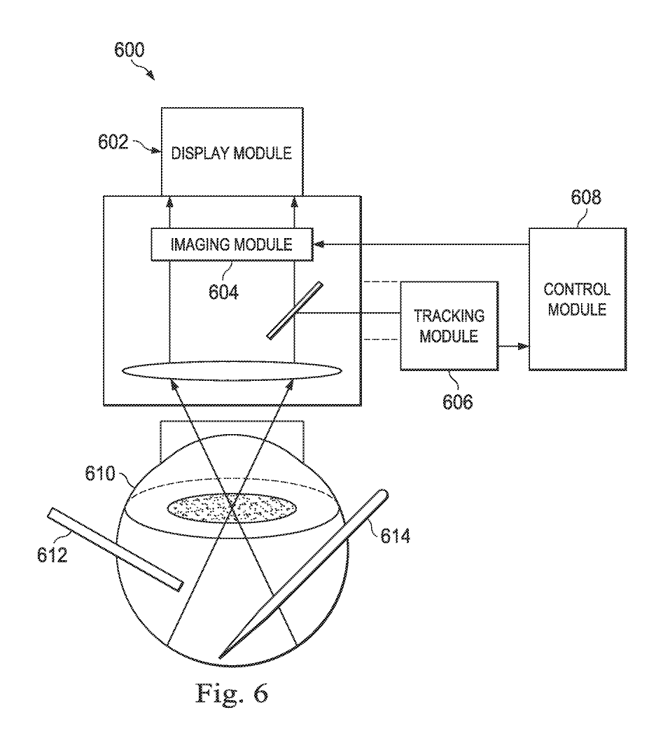

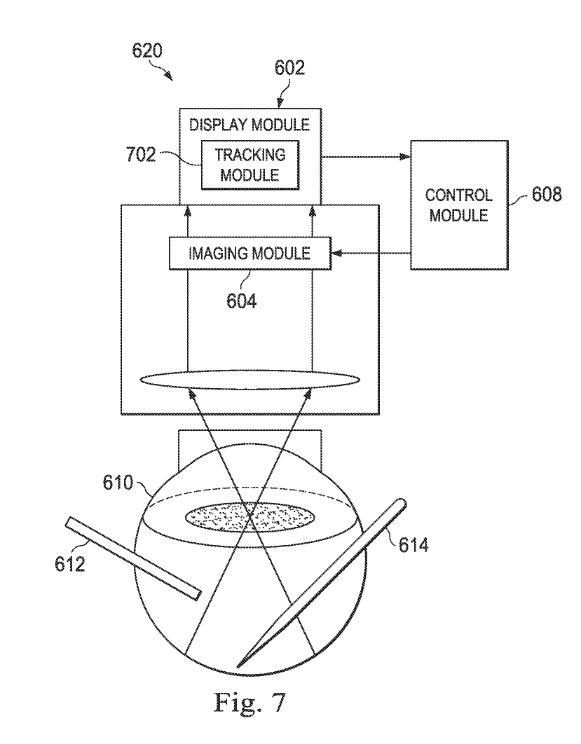

FIGS. 6 and 7 are diagrams that show imaging systems 600, 700 that use tool based tracking and eye tracking respectively to determine a current region of interest. FIG. 6 is a diagram showing an illustrative imaging system 600 that uses tool tracking. According to the present example, the imaging system includes a display module 602, an imaging module 604, a tracking module 606, and a control module 608. Any of these modules may form part of or utilize the control system 112 or other element of the system 100 of FIG. 1.

The imaging module 604 includes hardware, software, or a combination of both that is configured to obtain images of a surgical site such as the eye 610 of a patient. Included within such images may be various surgical tools 612, 614 such as an illuminator 612 and a forceps 614. The imaging module 604 may include a microscope imaging system (e.g., 106, FIG. 1) and an OCT imaging system (e.g. 108, FIG. 1). The imaging module 604 provides imaging data to the display module 602.

The display module 602 includes hardware, software, or a combination of both configured to display images to a user. Specifically, the display module 602 displays images obtained by the imaging module 604. Such images may include images of the surgical site as well as surgical data presented in an overlay as described above. The manner in which the surgical data overlay is presented may be based on instructions received from the control module 608. The display module 608 may correspond to the image viewer 104 described above.

The control module 608 includes hardware, software, or a combination of both configured to arrange the images obtained by the imaging module 604 for display by the display module 602. Specifically, the control module receives tracking data from the tracking module 604 that can be used to determine the current region of interest. In this example, the tracking module 606 tracks the location of the tool 614 within the image. Specifically, the tracking module 606 determines the location and/or orientation of the tool 614. Based on this information, a region of interest within the image can be inferred. For example, if the tip of the tool is moving around in a specific area, then the control module 608 defines a region of interest that encompasses that specific area. The control module 608 may correspond to the control system 112 described above.

FIG. 7 illustrates an imaging system 620 that includes a tracking module 702 designed to track the user's eyes. The tracking module 702 may form part of or utilize the control system 112 or other element of the system 100 of FIG. 1. The tracking module 702 is configured to detect where within an image being displayed by the display module 608 a user's eyes are being directed. The tracking module 702 can provide such information to the control module 608 for analysis. For example, if the user is viewing a specific region of the patient's eye 610, the control module 608 can determine a region of interest that encompasses that specific region.

Through use of principles described herein, a user can have a better experience when viewing the surgical site. Specifically, the user does not have to look at the top of the image every time he or she wishes to view the surgical data overlay. Rather, the surgical data overlay will be continually repositioned so that it is at a convenient position for the user.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

1. A method for display optimization, the method performed by a computing system, the method comprising:
receiving an image of a surgical site from an imaging system;
tracking an OCT beam that is used to generate an OCT image;
determining a region of interest at a first location within the image by determining a region of the image at which an OCT beam is directed;
generating a surgical data overlay at a first position, the first position associated with the first location of the region of interest;
detecting that the region of interest has moved to a second location within the image;
in response to detecting that the region of interest has moved to the second location, moving the surgical data overlay to a second position, the second position associated with the second location; and
displaying the image and surgical data overlay to a user.

2. The method of claim 1, wherein determining the region of interest further comprises, with a tool tracking system, detecting a region within the image at which a specified portion of a tool is operating.

3. The method of claim 2, wherein the tool tracking system determines a location of the tool based on analysis of the image.

4. The method of claim 2, wherein detecting that the region of interest has moved to the second location comprises determining that the specified portion of the tool has moved to the second location.

5. The method of claim 1, wherein determining the region of interest further comprises detecting a region within the image at which the user's eyes are directed.

6. The method of claim 5, wherein detecting that the region of interest has moved to the second location comprises determining that the user's eyes have been redirected to the second location.

7. The method of claim 1, wherein the surgical data overlay comprises an Optical Coherence Tomography (OCT) image of the region of interest obtained by an OCT imaging system.

8. (canceled)

9. The method of claim 1, wherein determining the region of the image at which the OCT beam is directed comprises analyzing the image to detect light within an OCT spectrum.

10. The method of claim 1, wherein the imaging system comprises a microscope imaging system.

11. The method of claim 7, wherein the imaging system includes an endoprobe.

12. The method of claim 1, wherein surgical data of the surgical data overlay includes at least one of: a pre-scan diagnostic image of the surgical site, pathology data, hand drawn graphs, surgical parameters and an enhanced image of the surgical site that emphasizes at least one of: an internal limiting membrane (ILM), an epi-retinal membrane (ERM), a thickness of the ERM, a thickness of one or more retinal layers, flow velocity of one or more retinal vessels, retinal angiographic information, characteristic information of one or more retinal layers, a contour of the ERM, a sub-retinal fluid (SRF), a thickness of the (SRF), and a volume of the SRF.

13. The method of claim 1, further comprising overlaying the surgical data with respect to the region of interest based on user preferences.

14. The method of claim 1, wherein the surgical site comprises a portion of a retina.

15. A system comprising:
an imaging module to obtain an image of a surgical site;
a display module to:
display the image of the surgical site to a user; and
display surgical data overlaying the image of the surgical site;
a tracking module to determine a region of interest of the surgical site at a first location by tracking an OCT beam to determine a region of the image at which the OCT beam is directed; and
a control module to:
detect that the region of interest has moved to a second location based on data from the tracking module; and
instruct the display module to move the surgical data to a new position over the image based on the new region of interest.

16. The system of claim 15, wherein the tracking module is further configured to detect the region of interest based on at least one of: tracking a tool within the image and determining where at the image the user's eyes are directed.

17. The system of claim 15, wherein the surgical data comprises an OCT image at the region of interest.

18. A method for display optimization, the method performed by a computing system, the method comprising:
receiving an image of a surgical site from an imaging system;
determining a region of interest within the image;
generating a surgical data overlay at a first position in the image, the first position associated with a first location of the region of interest, the surgical data overlay comprising an Optical Coherence Tomography (OCT) image of the region of interest;
tracking an OCT beam that is used to generate the OCT image;
detecting that the region of interest has moved to a second location by determining a region of the image at which the OCT beam is directed;
in response to detecting that the region of interest has moved to the second location, determining a second position for the surgical data overlay in the image based on both user preferences and the second location of the region of interest; and
displaying the image and surgical data overlay at the second position to a user.

19. The method of claim 18, wherein determining the region of interest and determining the region of interest has moved to a second location further comprises one of: detecting a location of a tool that is present within the image and detecting a region of the image at which a user's eyes are directed.

20. The method of claim 18, wherein the OCT image is acquired from an OCT imaging system that includes an endoprobe.
